(19) United States
(12) Patent Application Publication
(10) Pub. No.: US 2008/0028642 A1
(43) Pub. Date: Feb. 7, 2008

(54) Multiple Structure Shock Absorbing Members

(76) Inventor: Chol Hong Kim, Siheung-si (KR)

Correspondence Address: IPLA P.A., 3580 WILSHIRE BLVD., 17TH FLOOR, LOS ANGELES, CA, 90010, US

(21) Appl. No.: 11/569,121

(22) PCT Filed: Aug. 29, 2005
(86) PCT No.: PCT/KR05/02851

§ 371 Date: Nov. 14, 2006

(30) Foreign Application Priority Data
Aug. 30, 2004 (KR) 10-2004-0068501
Nov. 8, 2004 (KR) 10-2004-0090313

(51) Int. Cl. A61F 5/14

(52) U.S. Cl. 36/140

(57) ABSTRACT

A shock-absorbing member of the present invention is a multiple structure-shock absorbing member having the structure in which upper circular protrusions, repeatedly arranged at regular intervals in the right-left direction and in the upper-down direction, are connected to each other by cross-shaped connection ribs, which are connected to the lower sides of the upper circular protrusions in a rounded shap, and spacing holes are formed between the upper circular protrusions neighboring with each other The multiple structure-shock absorbing members provide finger-pressure therapy function and ventilation function and absorb and mitigate shock in multiple steps when a strong shock is applied to a small space from the exterior

### TECHNICAL FIELD

The present invention relates to a multiple structure-shock absorbing member, and more particularly, to a multiple structure-shock absorbing member having finger-pressure therapy function and ventilation function and capable of absorbing and mitigating shock in multiple steps when a strong force is applied to a small space from the exterior.

### BACKGROUND ART

Generally, there are various shock absorbing members applied to pads and shoe insoles. For example, shock-absorbing members applied to shoe insoles mainly have a lattice structure in which air is filled or a duplicate net structure.

However, the conventional shock-absorbing member applied to the shoe insole does not exhibit sufficient shock absorption because the structure is very complicated; due to weakened durability

### DISCLODURE OF INVENTION

### Technical Problem

Therefore, the present invention has been made in view of the above problems, and it is an object of the present invention to provide a multiple structure-shock-absorbing member having a structure, in which circular protrusions are connected to each other by connection ribs and repeatedly arranged, and spacing holes are formed between the circular protrusions, and functions of sufficiently and continuously absorbing shock, of finger-pressure, and of ventilating air.

### Technical Solution

In accordance with first and second preferred embodiments of the present invention, the above and other objects can be accomplished by the provision of a multiple structure-shock absorbing member shock-absorbing member for mitigating external shock and vibration, including: upper circular protrusions, repeatedly arranged at regular intervals in the right-left direction and in the upper-down direction, and connected to each other by cross-shaped connection ribs, which are connected to the lower sides of the upper circular protrusions in a rounded shape, or by lower circular protrusions having the cross-shaped connection ribs; and spacing holes formed between the upper circular protrusions neighboring with each other.

In accordance with third and fourth preferred embodiments of the present invention, the above and other objects can be accomplished by the provision of a multiple structure-shock absorbing member shock-absorbing member for mitigating external shock and vibration, including: upper circular protrusions, repeatedly arranged at regular intervals, and connected to each other by cross-shaped connection ribs, which are connected to the lower sides of the upper circular protrusions in a rounded shape; spacing holes formed between the upper circular protrusions neighboring with each other; contact protrusions, integrally formed with the lower sides of the upper circular protrusions or of the cross-shaped connection ribs, of which the height 1 is longer than the thickness t of the cross-shaped connection ribs.

### Advantageous Effects

As described above, since the multiple structure-shock absorbing members according to the present invention is made of flexible and soft material such as urethane, when external impact load is applied, the multiple structure-shock absorbing member absorbs the external impact load in multiple steps and is easily manufactured in commercial quantity.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a partial plan view illustrating a shock-absorbing member according to a first preferred embodiment of the present invention;

FIG. 2 is an enlarged plan view of the portion “A” of FIG. 1;

FIG. 3 is a sectional view taken along the line I-I of FIG. 2;

FIG. 4 is a partially enlarged plan view, similar to FIG. 2, illustrating a shock-absorbing member according to a second preferred embodiment of the present invention;

FIG. 5 is a plan model picture illustrating a shoe insole made of the shock-absorbing member according to the first preferred embodiment of the present invention;

FIG. 6 is a plan model picture illustrating a shoe insole made of the shock-absorbing member according to the second preferred embodiment of the present invention;

FIG. 7 is a partially enlarged plan view, similar to FIG. 2, illustrating a shock-absorbing member according to a third preferred embodiment of the present invention;

FIGS. 8 and 9 are sectional views taken along the line II-II of FIG. 7;

FIG. 10 is a partially enlarged plan view, similar to FIG. 8, illustrating a shock-absorbing member according to a fourth preferred embodiment of the present invention;

FIG. 11 is a sectional view taken along the line III-III of FIG. 10; and

FIG. 12 is a plan model picture illustrating a shoe insole made of the shock-absorbing member according to the fourth preferred embodiment of the present invention.

### BEST MODE FOR CARRYING OUT THE INVENTION

Hereinafter, a shock-absorbing member according to the present invention will be described in detail with reference to the accompanying drawings.

The shock-absorbing member according to the present invention can be applied to shoe insoles or shoe outsoles and other similar technical fields, and can be made of a soft and flexible material such as urethane.

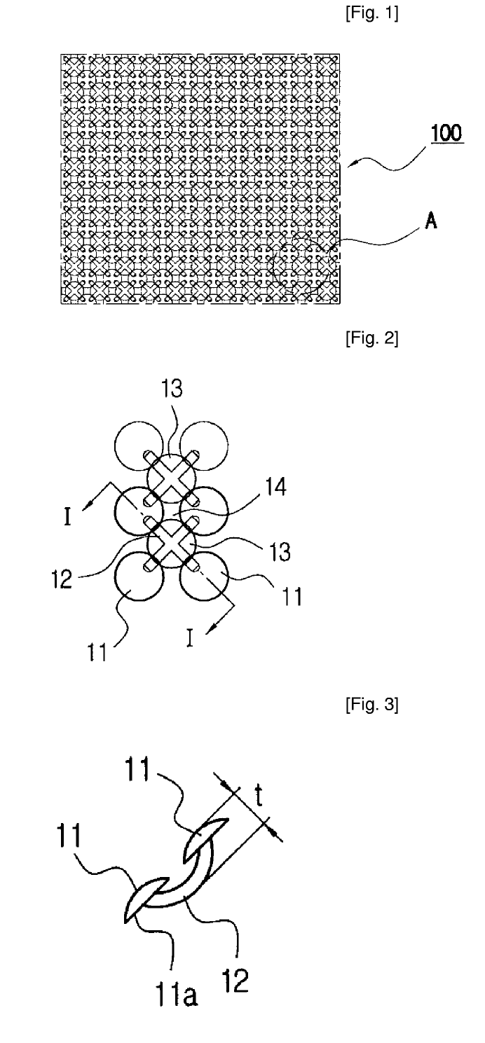

FIG. 1 is a partial plan view illustrating a shock-absorbing member 100 according to a first preferred embodiment of the present invention, FIG. 2 is an enlarged plan view of the portion “A” of FIG. 1, and FIG. 3 is a sectional view taken along the line I-I of FIG. 2.

The shock-absorbing member 100 according to the first preferred embodiment of the present invention, as shown in FIG. 1, is a member for absorbing and mitigating external shock and noise, and has a structure in which upper circular protrusions 11, repeatedly arranged at regular intervals in the right-left direction and in the upper-down direction, are connected to each other by upper circular protrusions 13 having cross-shaped connection ribs 12, the cross-shaped connection ribs 12 are connected to the lower sides 1 la of the upper circular protrusions 11 in a rounded shape, and spacing holes 14 are formed between the upper circular protrusions 11 neighboring with each other.

Thus, the upper sides in which the upper circular protrusions 11 are arranged and the lower side in which the cross-shaped connection ribs 12 for connecting the upper circular protrusions 11 is arranged have a predetermined thickness t as shown in FIG. 3.

In the shock-absorbing member 100 according to the first preferred embodiment of the present invention, when external force is applied to the upper side of the shock-absorbing member 100 in which the upper circular protrusions 11 are arranged, the external force pushes the upper circular protrusions 11 so that positions of the upper circular protrusions 11 are changed by the cross-shaped connection ribs 12 to absorb and mitigate shock. Moreover, since the spacing holes 14, formed between the upper circular protrusions 11, serve as ventilation holes, air is ventilated.

### Mode for the Invention

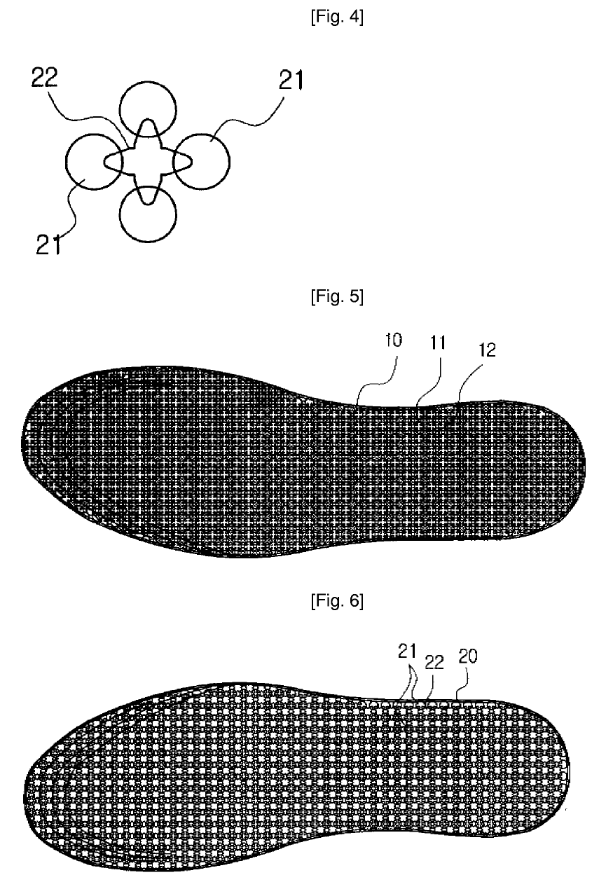

Among the accompanying drawing, FIG. 4 is similar to FIG. 3, and is a partially enlarged plan view illustrating a shock-absorbing member according to a second preferred embodiment of the present invention. FIG. 4 shows the structure in which rounded cross-shaped connection ribs 22, formed on the lower sides 21 of upper circular protrusions 21, connect the upper circular protrusions 21, arranged on the upper side of the shock-absorbing member of the second preferred embodiment of the present invention, to each other. In this preferred embodiment, the cross-shaped connection ribs 22 have a shape and an appearance different from those of the cross-shaped connection ribs 12 of FIGS. 1 and 2, but have the same operation and effect as those of the cross-shaped connection ribs 12.

Among the accompanying drawings, FIG. 5 is a plan model picture illustrating a shoe insole 10 made of the shock-absorbing member according to the first preferred embodiment of the present invention, and FIG. 6 is a plan model picture illustrating a shoe insole 20 made of the shock-absorbing member according to the second preferred embodiment of the present invention. As shown in the drawing, in the shoe insoles 10 and 20 made of the shock-absorbing member according to the first and second preferred embodiments of the present invention, air is ventilated through the spacing holes 14 and 24, the upper circular protrusions 11 and 21 and the cross-shaped connection ribs mitigate shock applied from the exterior to the sole of a foot and partially provide finger-pressure therapy effect to the sole of a foot.

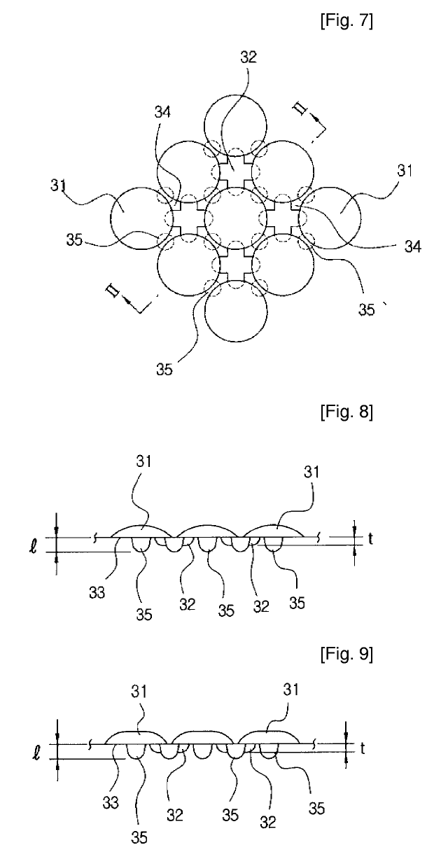

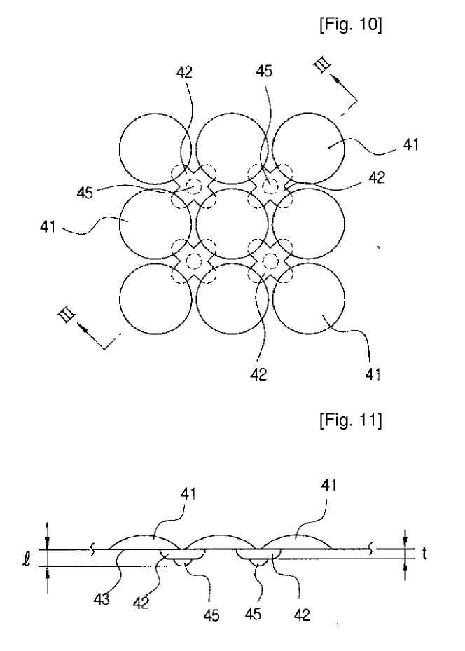

Among the accompanying drawings, FIG. 7 is a partially enlarged plan view, similar to FIG. 2, illustrating a shock-absorbing member according to a third preferred embodiment of the present invention, and FIG. 10 is a partially enlarged plan view, similar to FIG. 7, illustrating a shock-absorbing member according to a fourth preferred embodiment of the present invention. As shown in FIGS. 7 and 10, the shock-absorbing member according to the third and fourth preferred embodiments of the present invention have the structure in which upper circular protrusions 31 and 41 are repeatedly and continuously arranged at regular intervals by cross-shaped connection ribs 32 and 42 for connecting the lower sides 31a and 41a of the upper circular protrusions 31 and 41, the cross-shaped connection ribs 32 and 42 connect the lower sides 31 a and 41 a of the upper circular protrusions 31 and 41 in a rounded shape, and spacing holes 34 and 44 are formed between the upper circular protrusions 31 and 41 neighboring with each other.

Among the preferred embodiments, in the third preferred embodiment, contact protrusions 35 are formed on the lower sides 3 la of the upper circular protrusions 31 neighboring with each other and the height 1 of the contact protrusions 35 of the lower sides 3 la, as shown in FIG. 7, is longer than the thickness t of the cross-shaped connection ribs 32.

Additionally, according to the fourth preferred embodiment of the present invention, contact protrusions 45 are integrally formed with the central regions of the cross-shaped connection ribs 42 for connecting the lower sides 41a of the upper circular protrusions 41, and in this preferred embodiment, as shown in FIG. 11 showing a sectional view taken along the line III-III of FIG. 8, it can be understood that the height of the contact protrusions 45 is longer than the thickness of cross-shaped connection ribs 42.

Among the accompanying drawings, FIGS. 8 and 9 are sectional views taken along the line II-II of FIG. 7, wherein FIG. 8 illustrates that the upper circular protrusions 31 having convex upper sides of a perfect semi-circular cross-section and FIG. 9 illustrates that the upper circular protrusions 31 having convex upper sides of a flat cross-section.

Thus, in the shock-absorbing members according to the third and fourth preferred embodiments of the present invention, when external loads are applied to the upper circular protrusions 31 and 41, the contact protrusions 33 and 45, formed on the lower sides 31a and 41a and having the long heights 1, primarily absorb and mitigate shock, and the cross-shaped connection ribs 32 and 42, having the thickness t thinner than the height of the contact protrusions 33 and 45, secondly absorb and mitigate shock.

Although the contact protrusions 35 and 45 of the third and fourth preferred embodiments of the present invention are disposed at different positions on the upper circular protrusions, the operation and effect thereof are similar to those as described above.

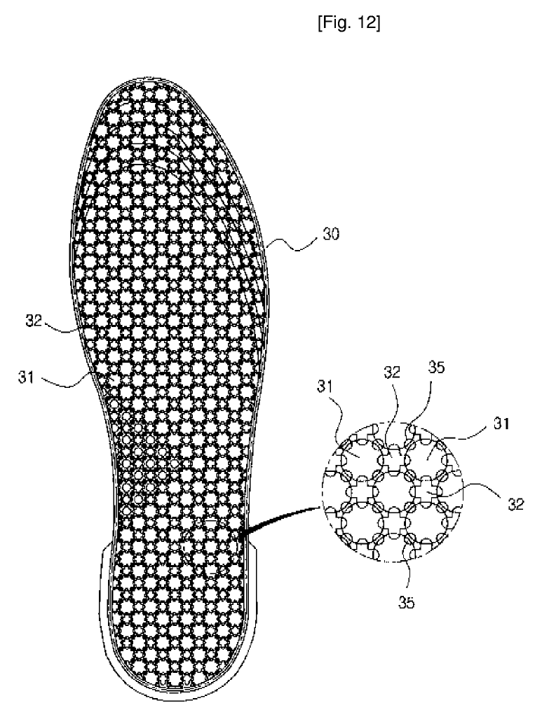

Among the accompanying drawings, FIG. 12 is a plan model picture illustrating a shoe insole 30 made of the shock-absorbing member according to the third preferred embodiment of the present invention. The shock-absorbing member can be widely applied to general pads, mats, and shock-mitigating products for absorbing shock as the shoe insole.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

As described above, since the multiple structure-shock absorbing members according to the present invention is made of flexible and soft material such as urethane, when external impact load is applied, the multiple structure-shock absorbing member absorbs the external impact load in multiple steps and is easily manufactured in commercial quantity.

1. A shock-absorbing member for mitigating external shock and vibration, comprising:
upper circular protrusions. repeatedly arranged at regular intervals in the right-left direction and in the upper-down direction, and connected to each other by cross-shaped connection ribs, which are connected to the lower sides of the upper circular protrusions in a rounded shape; and
spacing holes formed between the upper circular protrusions neighboring with each other.

2. The shock-absorbing member according to claim 1, wherein the upper circular protrusions are connected to each other by lower circular protrusions having the cross-shaped connection ribs.

3. The shock-absorbing member according to claim 1, wherein the upper sides in which the upper circular protrusions are arranged and the lower side in which the cross-shaped connection ribs for connecting the upper circular protrusions is arranged have a predetermined thickness t.

4. The shock-absorbing member according to claim 1, wherein contact protrusions are formed on the lower sides of the upper circular protrusion and other upper circular protrusions neighboring with the upper circular protrusion.

5. The shock-absorbing member according to claim 1, wherein contact protrusions are integrally formed with the central regions of the cross-shaped connection ribs.

6. The shock-absorbing member according to elaim 4, wherein the height (l) of the contact protrusions is longer than the thickness t of the cross-shaped connection ribs.

7. The shock-absorbing member according to any one claim 1, wherein the upper circular protrusions have convex upper sides of a perfect semi-circular cross-section or convex upper sides of a flat cross-section.

8. The shock-absorbing member according to claim 2, wherein the upper sides in which the upper circular protrusions are arranged and the lower side in which the cross-shaped connection ribs for connecting the upper circular protrusions is arranged have a predetermined thickness t.

9. The shock-absorbing member according to claim 5, wherein the height (l) of the contact protrusions is longer than the thickness t of the cross-shaped connection ribs.
